(19) United States
(12) Patent Application Publication
(10) Pub. No.: US 2024/0376130 A1
(43) Pub. Date: Nov. 14, 2024

(54) SINGLET OXYGEN CAPTURING OR RELEASING MATERIAL, PREPARATION METHOD AND USE THEREOF

(71) Applicant: SOOCHOW UNIVERSITY, Wujiang (CN)

(72) Inventors: Jianping LANG, Wujiang (CN); Yu Ge, Wujiang (CN); Qiuyi Li, Wujiang (CN); Chunyan Ni, Wujiang (CN)

(21) Appl. No.: 18/673,187

(22) Filed: May 23, 2024

Related U.S. Application Data
(63) Continuation of application No. PCT/CN2023/123697, filed on Oct. 10, 2023.

(30) Foreign Application Priority Data
May 10, 2023 (CN) 202310519205.X

(51) Int. Cl. C07F 3/00; G01N 21/64; G01N 33/00

(52) U.S. Cl. CPC C07F 3/00; G01N 21/6428; G01N 33/0027

(57) ABSTRACT

The present invention relates to the field of singlet oxygen technologies, and particularly to a singlet oxygen capturing or releasing material, and a preparation method and use thereof. The singlet oxygen capturing material according to the present invention has a chemical formula of [Cd(BP4VA)(4-NBA)₂]ₙ; and the singlet oxygen releasing material has a chemical formula of [Cd(BP4VA-¹O₂)(4-NBA)₂]ₙ, in which BP4VA is 9,10-bis[(cis)-2-(pyridin-4-yl)vinyl]anthracene, 4-NBA is a unit derived by removing a hydrogen ion from 4-nitrobenzoic acid after reaction, and n=3000-50000. The material has simple synthesis steps and high yield, and is capable of being prepared in large quantities. The material is capable of efficiently and rapidly capturing or releasing singlet oxygen. The singlet oxygen capturing material is also useful as a fluorescence sensor for detecting oxygen, with the advantages of simple operation, high selectivity, high sensitivity, and good recyclability and good stability.

This application is a Continuation Application of PCT/CN2023/123697, filed on Oct. 10, 2023, which claims priority to Chinese Patent Application No. 202310519205.X, filed on May 10, 2023, which is incorporated by reference for all purposes as if fully set forth herein.

### FIELD OF THE INVENTION

The present invention relates to the field of singlet oxygen technologies, and particularly to a singlet oxygen capturing or releasing material, and a preparation method and use thereof.

### DESCRIPTION OF THE RELATED ART

Singlet oxygen (¹O₂) is molecular oxygen in the excited state, which plays an important role in reactive oxygen species and is widely used in organic synthesis, photodynamic therapy and other fields (Y. Y. Liu, A. J. Howarth, J. T. Hupp, O. K. Farha, Angew. Chem. Int. Ed. 2015, 54, 9001). ¹O₂ has a short life (half-life: 0.03 ms-0.18 ms) (M. L. Liu, Y. C. Chen, Y. Guo, H. Yuan, T. X. Cui, S. K. Yao, S. X. Jin, H. H. Fan, C. J. Wang, R. Xie, W. J. He, Z. J. Guo, Nat. Commun. 2022, 13, 2179), so it is particularly important to capture and release ¹O₂ under controllable conditions. The most commonly used method for producing ¹O₂ is that a photosensitizer promotes the energy transfer from excited triplet O₂(³O₂) to and formation of ¹O₂ under light irradiation (R. W. Redmond, J. N. Gamlin, Photochem. Photobio. 1999, 70, 391). The commonly used photosensitizers include organic dyes such as methylene blue and rose red. Generally, such organic dyes tend to aggregate at a high concentration, and fade under long-term light irradiation, leading to a decreased sensitizing ability. In recent years, anthracene or anthracene ring in its derivatives is reacted with a dienophile molecule through [4+2] cycloaddition, also called Diels-Alder reaction (V. N. Huynh, M. Leitner, A. Bhattacharyya, L. Uhlstein, P. Kreitmeier, P. Sakrausky, J. Rehbein, O. Reiser, Commun. Chem., 2020, 3, 158). Singlet oxygen is a special dienophile molecule, which can react with anthracene and its derivatives to form a corresponding endoperoxide. The endoperoxide can react reversibly when irradiated by ultraviolet rays or heated to release singlet oxygen. So far, most photo-oxidation products of anthracene and its derivatives need to be heated or irradiated for more than several hours before they can undergo a reversible reaction to release ¹O₂ (H. W. Lai, J. Y. Yan, S. Liu, Q. Z. Yang, F. Y. Xing, P. Xiao, Angew. Chem. Int. Ed. 2020, 59, 10431). Currently, the photo-oxidation process of anthryl compounds is mainly carried out in solution under ultraviolet light. The reaction is largely limited to organic substances, and covalent organic polymers and so on. There are few examples of coordination polymers (CPs) in solid that can effectively capture and release ¹O₂.

CPs are a crystalline material formed by the connection of a metal node and an organic ligand through a coordination bond, which have attracted more and more attention. They are widely used in gas storage and separation, chemical and biological sensing, catalysis and many other fields (W. Fudickar, T. Linker, J. Am. Chem. Soc. 2012, 134, 15071; C. Mongin, A. M. Ardoy, R. Mereau, D. M. Bassani, B. Bibal, Chem. Sci. 2020, 11, 1478). The use of an anthryl compound with excellent luminescence and photoresponsibility as an organic ligand to assemble CPs with rich topological structures may open up a new way for Cps materials to capture and release ¹O₂ efficiently and quickly. Therefore, it is of great significance to design and synthesize a coordination polymer containing an anthracene ligand, and to greatly improve the yield of singlet oxygen in a green, clean and efficient way.

### SUMMARY OF THE INVENTION

To this end, the technical problems to be solved in the present invention are to overcome those such as low singlet oxygen capturing or releasing efficiency of materials, complicated synthesis method and the like in the prior art.

To solve the above technical problems, the present invention provides a singlet oxygen capturing or releasing material, and a preparation method and use thereof. The material has simple synthesis steps and high yield, and is capable of being prepared in large quantities; and is a material capable of efficiently and rapidly capturing or releasing singlet oxygen. The singlet oxygen capturing material is also useful as a fluorescence sensor for detecting oxygen, with the advantages of simple operation, high selectivity, high sensitivity, and good cycling performance and good stability.

A first object of the present invention is to provide a singlet oxygen capturing or releasing material. The singlet oxygen capturing material has a chemical formula of [Cd(BP4VA)(4-NBA)₂]ₙ; and the singlet oxygen releasing material has a chemical formula of [Cd(BP4VA-¹O₂)(4-NBA)₂]ₙ, in which BP4VA is 9,10-bis[(cis)-2-(pyridin-4-yl)vinyl]anthracene, 4-NBA is a unit derived by removing a hydrogen ion from 4-nitrobenzoic acid (4-HNBA) after reaction, and n=3000-50000.

In an embodiment of the present invention, the singlet oxygen capturing material [Cd(BP4VA)(4-NBA)₂]ₙ is a Cd₂(4-NBA)₄-based one-dimensional double chain material, which is a one-dimensional coordination polymer constructed with Cd₂(4-NBA)₄ as a connection node and 9,10-bis[(cis)-2-(pyridin-4-yl)vinyl]anthracene as a bridging ligand.

In an embodiment of the present invention, the singlet oxygen releasing material [Cd(BP4VA-¹O₂)(4-NBA)₂]ₙ is a Cd₂(4-NBA)₄-based one-dimensional double chain material, which is a one-dimensional coordination polymer constructed with Cd₂(4-NBA)₄ as a connection node and 9,10-bis[(cis)-2-(pyridin-4-yl)vinyl-peroxy]anthracene as a bridging ligand.

A second object of the present invention is to provide a method for preparing the singlet oxygen capturing or releasing material, which includes the following steps:
S1: dissolving a soluble cadmium salt, 9,10-bis[(cis)-2-(pyridin-4-yl)vinyl]anthracene, and 4-nitrobenzoic acid in a solvent, reacting with heating, and filtering after the reaction, to obtain the singlet oxygen capturing material [Cd(BP4VA)(4-NBA)₂]ₙ; and
S2: subjecting the singlet oxygen capturing material obtained in S1 to a photo-oxidation reaction under an oxygen atmosphere, to obtain the singlet oxygen releasing material [Cd(BP4VA-¹O₂)(4-NBA)₂]ₙ;
in which BP4VA is 9,10-bis[(cis)-2-(pyridin-4-yl)vinyl]anthracene, 4-NBA is a unit derived by removing a hydrogen ion from 4-nitrobenzoic acid after reaction, and n=3000-50000.

In an embodiment of the present invention, in S1, the soluble cadmium salt is selected from the group consisting of cadmium nitrate, cadmium sulfate, cadmium perchlorate, cadmium carbonate and any combination thereof.

In an embodiment of the present invention, in S1, the molar ratio of the soluble cadmium salt, 9,10-bis[(cis)-2-(pyridin-4-yl)vinyl]anthracene and 4-nitrobenzoic acid is 1-1.5:0.75-1.5:1.5-2.5.

In an embodiment of the present invention, in S1, the reaction temperature is 120° C.-135° C., and the reaction time is 18-36 h.

In an embodiment of the present invention, in S1, the solvent includes N,N-dimethyl acetamide, acetonitrile and water mixed in a volume ratio of 1.0-2.0:1.0-2.0:2.5-3.5.

In an embodiment of the present invention, after S1, the method further includes a step of washing and drying the product.

In an embodiment of the present invention, in S2, the light source for the photo-oxidation reaction is visible light having a wavelength of 475 nm.

In an embodiment of the present invention, in S2, the photo-oxidation reaction is a [4+2] photo-oxidation reaction.

In an embodiment of the present invention, in S2, the photo-oxidation reaction time is 4-7 h.

In an embodiment of the present invention, in S2, the oxygen is excessive, and [Cd(BP4VA)(4-NBA)₂]ₙ sensitizes the oxygen to produce singlet oxygen.

A third object of the present invention is to provide a fluorescence sensor, which includes the singlet oxygen capturing material.

A fourth object of the present invention is to provide use of the fluorescence sensor in the detection of oxygen.

Compared with the prior art, the technical solution of the present invention has the following advantages.

(1) The singlet oxygen capturing or releasing material of the present invention is a one-dimensional coordination polymer material based on the Cd₂(4-NBA)₄ unit, which can be obtained by a relatively simple synthesis process. In its structure, Cd₂(4-NBA)₄ is used as a node, and 9,10-bis[(cis)-2-(pyridin-4-yl)vinyl]anthracene is used as a bridging ligand, where the anthracene ring serving as a conjugated diene with 4π electrons is reacted with a dienophile with 2π electrons by [4+2] cycloaddition, also called Diels-Alder reaction. In the Diels-Alder reaction, anthracene and its derivatives are typical conjugated dienes, and singlet oxygen is a special dienophile, which makes it possible for anthracene and its derivatives to undergo reversible [4+2] cycloaddition with ¹O₂.

(2) The singlet oxygen capturing or releasing material of the present invention can sensitize O₂ to form ¹O₂, and quickly capture the formed ¹O₂. The obtained product can release ¹O₂ quickly under the irradiation of microwave.

(3) The singlet oxygen capturing or releasing material of the present invention shows a very sensitive response speed in the fluorescence response experiment for oxygen, the response time is less than 5 s, and the recovery time is about 20 s-40 s. Moreover, the fluorescent material disclosed in the present invention has high stability, the response time and sensitivity have no obvious decline after 5 cycles, and the structure can remain stable in the air for 6 months without obvious disintegration.

### BRIEF DESCRIPTION OF THE DRAWINGS

To make the disclosure of the present invention more comprehensible, the present invention will be further described in detail by way of specific embodiments of the present invention with reference the accompanying drawings, in which:

FIG. 1 shows the coordination environment in a singlet oxygen capturing or releasing material according to the present invention, in which (a) shows the coordination environment of Cd1 in [Cd(BP4VA)(4-NBA)₂]ₙ, and (b) shows the coordination environment of Cd1 in [Cd(BP4VA-¹O₂)(4-NBA)₂]ₙ;

FIG. 2 shows a one-dimensional dual-chain singlet oxygen capturing or releasing material of the present invention, in which (a) is [Cd(BP4VA)(4-NBA)₂]ₙ, and (b) is [Cd(BP4VA-¹O₂)(4-NBA)₂]ₙ;

FIG. 3 shows a ¹H NMR spectrum (400 MHz, DMSO-d₆) of a singlet oxygen capturing or releasing material in Test Example 1 of the present invention, in which (a) is [Cd(BP4VA)(4-NBA)₂]ₙ, and (b) is [Cd(BP4VA-¹O₂)(4-NBA)₂]ₙ;

FIG. 4 shows a ¹³C NMR spectrum (400 MHz, DMSO-d₆) of the singlet oxygen capturing or releasing material in Test Example 1 of the present invention and the changes of the ligand BP4VA before and after photo-induced cycloaddition, in which (a) shows a ¹³C NMR spectrum of [Cd(BP4VA)(4-NBA)₂]ₙ, (b) shows a ¹³C NMR spectrum of [Cd(BP4VA-¹O₂)(4-NBA)₂]ₙ, and (c) shows the changes of the ligand BP4VA before and after photo-induced cycloaddition;

FIG. 5 shows an infrared spectrum of the singlet oxygen capturing or releasing material in Test Example 1 of the present invention, in which (a) is [Cd(BP4VA)(4-NBA)₂]ₙ, and (b) is [Cd(BP4VA-¹O₂)(4-NBA)₂]ₙ;

FIG. 6 shows an optical microscopy image of single crystalline [Cd(BP4VA)(4-NBA)₂]ₙ in Test Example 2 of the present invention before and after irradiation at 475 nm, in which (a) shows an optical microscopy image before irradiation, and (b) shows an optical microscopy image after irradiation;

FIG. 7 shows a ¹H NMR spectrum (400 MHz, DMSO-d₆) of [Cd(BP4VA)(4-NBA)₂]ₙ in Test Example 2 of the present invention after irradiation for various times;

FIG. 8 shows the test results of [Cd(BP4VA-¹O₂)(4-NBA)₂]ₙ in Test Example 3 of the present invention, in which (a) shows an ultraviolet-visible absorption spectrum of DPBF and [Cd(BP4VA-¹O₂)(4-NBA)₂]ₙ that are thermally decomposed to produce ¹O₂, and (b) compares the decline rates of DPBF (blue) alone and in the presence of [Cd(BP4VA-¹O₂)(4-NBA)₂]ₙ(red);

FIG. 9 is a schematic diagram of a fluorescence sensing device in Test Example 4 of the present invention;

FIG. 10 shows a fluorescence spectrum upon the reaction of [Cd(BP4VA)(4-NBA)₂]ₙ with O₂ and the changes (illustration) of the emission peak within 0-30 s in Test Example 4 of the present invention;

FIG. 11 shows a fluorescence spectrum when [Cd(BP4VA-¹O₂)(4-NBA)₂]ₙ releases ¹O₂ under microwave irradiation and the changes (illustration) of the emission peak within 0-120 s in Test Example 4 of the present invention;

FIG. 12 shows the changes of fluorescence intensity when [Cd(BP4VA)(4-NBA)₂]ₙ reacts with various concentrations of O₂ and a schematic diagram (illustration) showing the linear relationship between the fluorescence intensity and the concentration upon reaction with a low concentration of O₂ in Test Example 4 of the present invention;

FIG. 13 shows the changes of fluorescence intensity after 5 consecutive reversible photo-oxidation cycles in Test Example 5 of the present invention;

FIG. 14 shows a PXRD pattern of [Cd(BP4VA)(4-NBA)₂]ₙ after 5 consecutive reversible photo-oxidation cycles in Test Example 5 of the present invention;

FIG. 15 shows a PXRD pattern of [Cd(BP4VA)(4-NBA)₂]ₙ under various conditions in Test Example 6 of the present invention, in which a simulated pattern, a pattern of a synthesized sample, and a pattern after standing for three months and six months in the air are shown from bottom to top.

### DETAILED DESCRIPTION OF THE PREFERRED EMBODIMENTS

The present invention will be further described below with reference to the accompanying drawings and specific examples, so that those skilled in the art can better understand and implement the present invention; however, the present invention is not limited thereto.

### Example 1

The present invention relates to a singlet oxygen capturing or releasing material, and a preparation method and use thereof. The method comprises specifically the following steps:

S1: Preparation of singlet oxygen capturing material [Cd(BP4VA)(4-NBA)₂]ₙ:Cd(NO₃)₂·4H₂O (3.08 mg, 0.01 mmol), BP4VA (3.84 mg, 0.01 mmol) and 4-HNBA (3.35 mg, 0.02 mmol) were added to a thick-walled glass tube, and then 0.5 mL N,N-dimethyl acetamide (DMA), 0.5 mL of acetonitrile and 1 mL of water were added, sealed and ultrasonically dispersed for 5 min. The glass tube was positioned in a programmed oven, maintained at 120° C. for 24 h, and then cooled to 20° C. at a rate of 8° C./h. An orange bulk crystal [Cd(BP4VA)(4-NBA)₂]ₙ (n=3000-50000) was precipitated. The crystal was collected by filtration, washed thoroughly with anhydrous ethanol, and finally dried in the air, Yield: 0.0688 g (83%, based on BPV4A).

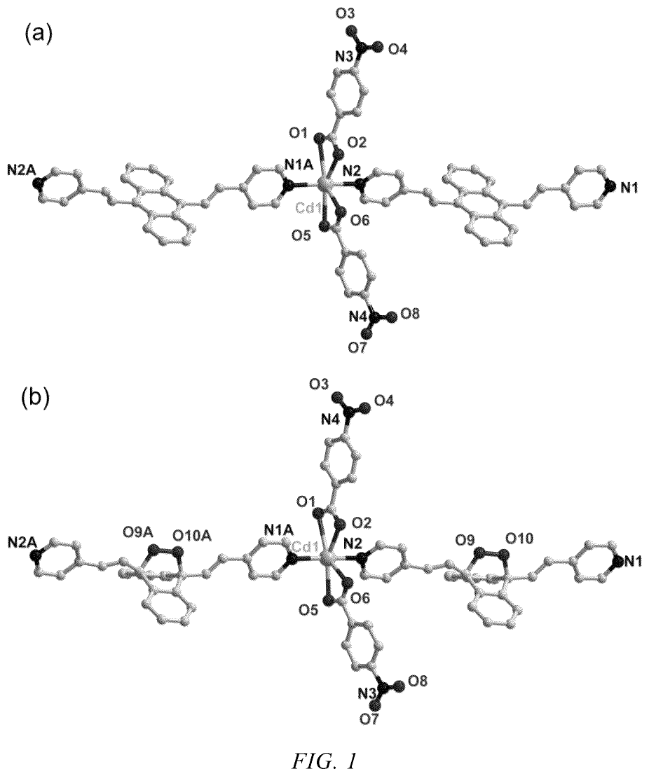

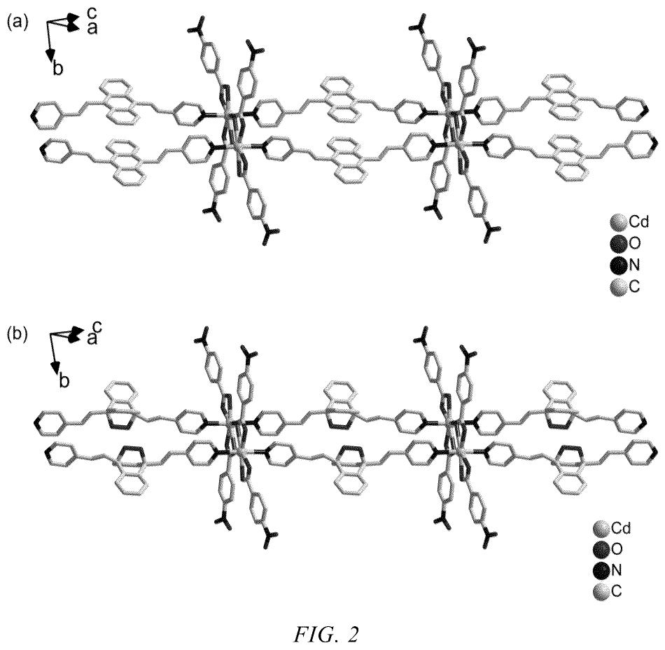

In [Cd(BP4VA)(4-NBA)₂]ₙ, the connection mode between the connection node Cd₂(4-NBA)₄ and the bridging ligand 9,10-bis[(cis)-2-(pyridin-4-yl)vinyl]anthracene is as shown in FIG. 1(a). A one-dimensional double chain structure is formed by the continuous extension in the direction of [1 0 1]. To better show the structure, the one-dimensional double chain structure is shown in FIG. 2(a).

S2: Preparation of singlet oxygen releasing material [Cd(BP4VA-¹O₂)(4-NBA)₂]ₙ: 200 mg of crystalline [Cd(BP4VA)(4-NBA)₂]ₙ was placed in a clean test tube filled with oxygen, and the crystalline [Cd(BP4VA)(4-NBA)₂]ₙ was irradiated for 5 h from 5 cm vertically directly above the test tube with an LED lamp (k=475 nm) with a power of 50 W (λ=475 nm), to obtain a 100% transformed [4+2] cycloaddition product, that is, the singlet oxygen releasing material [Cd(BP4VA-¹O₂)(4-NBA)₂]ₙ (n=3000-50000).

In [Cd(BP4VA-¹O₂)(4-NBA)₂]ₙ, the connection mode between the connection node Cd₂(4-NBA)₄ and the bridging ligand 9,10-bis[(cis)-2-(pyridin-4-yl)vinyl]peroxy-anthracene is shown in FIG. 1(b). A one-dimensional double chain structure is formed by the continuous extension in the direction of [1 0 1]. To better show the structure, the one-dimensional double chain structure is shown in FIG. 2(b).

### Test Example 1. Basic Characterization of [Cd(BP4VA)(4-NBA)₂]ₙ and [Cd(BP4VA-¹O₂)(4-NBA)₂]ₙ Coordination Polymer Materials

The singlet oxygen capturing or releasing material in Example 1 was characterized by nuclear magnetic resonance (NMR) spectroscopy, element analysis, infrared spectrometry and X-ray single crystal diffraction. The specific results are as follows:

### (1) NMR Spectrum:

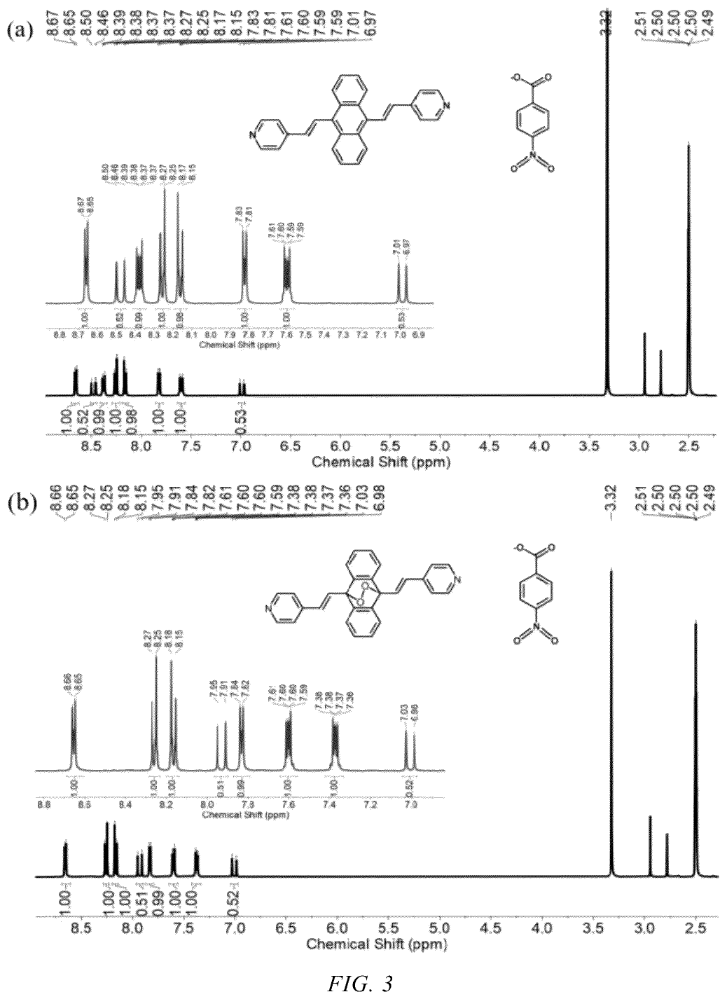

NMR hydrogen spectrum of singlet oxygen capturing material (FIG. 3a): ¹H-NMR (400 MHz, DMSO-d₆): δ 8.67 (d, 4H), 8.50 (d, 2H), 8.39 (m, 4H), 8.27 (d, 2H), 8.17 (d, 2H), 7.83 (d, 4H), 7.61 (m, 4H), 7.01 (d, 2H).

NMR hydrogen spectrum of singlet oxygen releasing material (FIG. 3b): ¹H-NMR (400 MHz, DMSO-d₆ ppm): δ 8.66 (d, 4H), 7.95 (d, 2H), 7.84 (d, 4H), 7.61 (m, 4H), 7.38 (m, 4H), 7.03 (d, 2H).

The NMR hydrogen spectrum shows that due to the formation of C—O bond, [Cd(BP4VA-¹O₂)(4-NBA)₂]ₙ has obvious chemically shifted absorption peak in the NMR hydrogen spectrum compared with [Cd(BP4VA)(4-NBA)₂]ₙ. NMR carbon spectrum of singlet oxygen capturing and releasing material (FIG. 4): ¹³C-NMR spectrum shows that for the photo-oxidation reaction [Cd(BP4VA-¹O₂)(4-NBA)₂]ₙ, a new characteristic peak appears at δ=81.7 ppm; and the signal is not observed in the ¹³C NMR spectrum of [Cd(BP4VA)(4-NBA)₂]ₙ, indicating that C—O bond is formed in the process of photo-induced cycloaddition.

### (2) Element Analysis (%):

Singlet oxygen capturing material C₄₂H₂₈N₄O₈Cd (M.W.=829.08), Calculated: C, 60.00; H, 3.95; N, 7.37%; Found: C, 60.64; H, 3.40; N, 6.76%.

Singlet oxygen releasing material C₄₂H₂₈N₄O₁₀Cd (M.W.=829.08), Calculated: C, 58.06; H, 3.47; N, 6.88%; Found: C, 58.58; H, 3.28; N, 6.51%.

Element analysis data shows that the found value and the calculated value for C, H, and N contents in the singlet oxygen capturing material C₄₂H₂₈N₄O₈Cd and the singlet oxygen releasing material C₄₂H₂₈N₄O₁₀Cd are consistent.

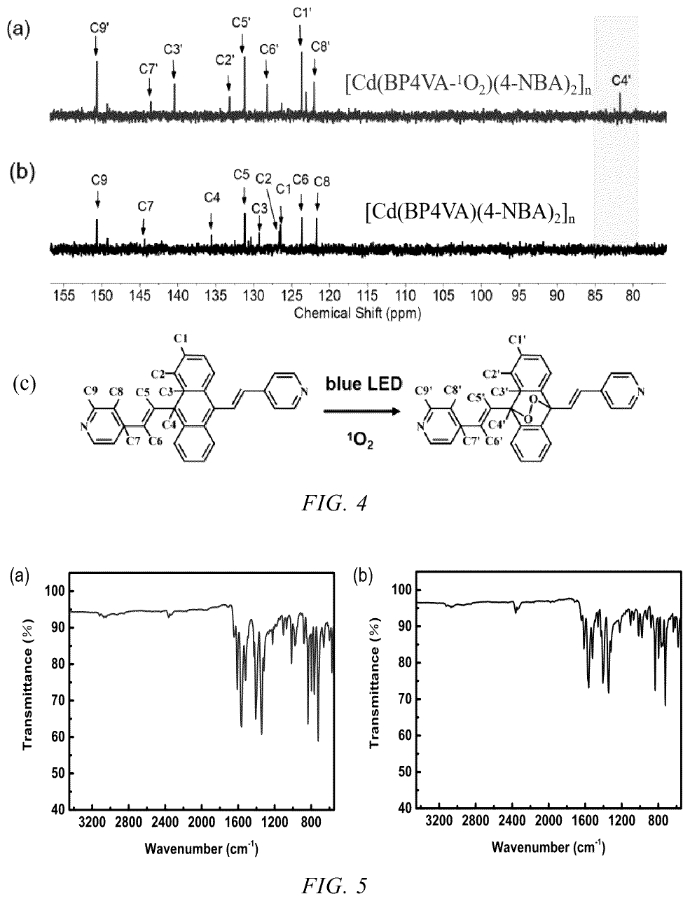

### (3) Infrared Spectrum (Fourier Transform) (FIG. 5):

IR spectrum of singlet oxygen capturing material, 1610 (s), 1562 (s), 1407 (s), 1342 (s), 1222 (w), 1104 (w), 1016 (m), 835 (s), 797 (s), 766 (s), 723 (s) cm⁻¹.

IR spectrum of singlet oxygen releasing material, 1613 (m), 1562 (s), 1406 (s), 1343 (s), 1221 (w), 1105 (w), 1015 (w), 978 (w), 834 (s), 796 (s), 723 (s), 636 (w) cm⁻¹.

### (4) X-Ray Single Crystal Diffraction:

The crystallographic parameters of compounds [Cd(BP4VA)(4-NBA)₂]ₙ and [Cd(BP4VA-¹O₂)(4-NBA)₂]ₙ are shown in Table 1:

TABLE 1

| | | [Cd(BP4VA-¹O₂) |
|---|---|---|
| Compounds | [Cd(BP4VA)(4-NBA)₂]ₙ | (4-NBA)₂]ₙ |
| Molecular | C₄₂H₂₈N₄O₈Cd | C₄₂H₂₈N₄O₁₀Cd |
| formula | | |
| Molecular weight | 829.08 | 861.08 |
| Crystal system | Trigonal system | Trigonal system |
| Space group | P1̅ | P1̅ |
| a/Å | 10.1359(6) | 10.0134(11) |
| b/Å | 13.7494(8) | 14.0021(15) |
| c/Å | 15.8519(9) | 15.7051(17) |
| α/° | 78.555(2) | 76.784(3) |
| β/° | 79.008(2) | 79.471(4) |
| γ/° | 89.584(2) | 88.066(4) |
| V/Å³ | 2124.4(2) | 2107.5(4) |
| Z | 2 | 2 |
| D_{c}/g cm⁻³ | 1.296 | 1.357 |
| μ (Mo—Kα)/mm⁻¹ | 0.567 | 0.577 |
| Total number of | 104239 | 79892 |
| diffraction points | | |
| Number of | 9855 | 7404 |
| independent | | |
| diffraction points | | |
| F(000) | 840 | 872 |
| R₁^{a} | 0.0471 | 0.0877 |
| wR₂^{b} | 0.0992 | 0.2922 |
| GOF^{c} | 1.036 | 1.074 |

The X-ray single crystal diffraction data in Table 1 shows that coordination polymer materials, namely, [Cd(BP4VA)(4-NBA)₂]ₙ and [Cd(BP4VA-¹O₂)(4-NBA)₂]ₙ are successfully obtained in Example 1.

### Test Example 2. Singlet Oxygen Capturing Test

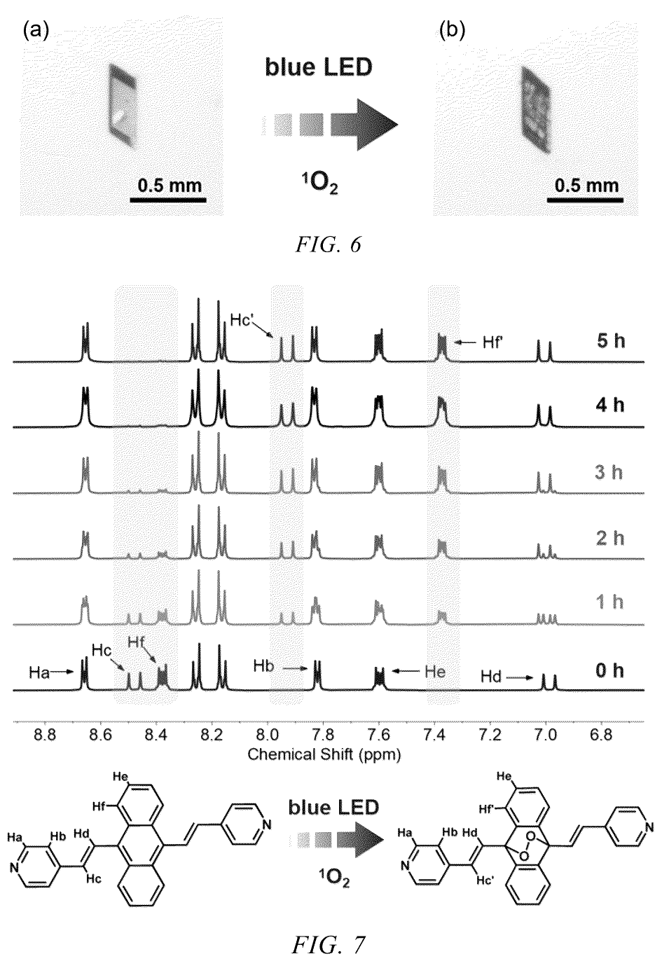

An optical microscopy image of single crystalline [Cd(BP4VA)(4-NBA)₂]ₙ before and after irradiation with visible light at 475 nm is shown in FIG. 6. As can be seen from FIG. 6, the crystalline [Cd(BP4VA)(4-NBA)₂]ₙ is ground into a very fine powder, and the powder can be fully reacted with the sensitized ¹O₂ under irradiation. The photo-induced cycloaddition reaction during the conversion of [Cd(BP4VA)(4-NBA)₂]ₙ into [Cd(BP4VA-¹O₂)(4-NBA)₂]ₙ was monitored by ¹H-NMR, and the reaction process at various irradiation times was tracked by ¹H-NMR. The results are shown in FIG. 7. As can be seen from FIG. 7, the proton signals of anthracene and olefin (δ=8.37 and 8.49 ppm) gradually decrease with the extension of irradiation time. Two groups of proton signals of the peroxide (δ=7.61 and 7.95 ppm) are gradually formed and finally reach the maximum. After 5 h, reaction is almost complete. The ¹H-NMR spectrum shows that the conversion is 100%.

### Test Example 3. Singlet Oxygen Releasing Test

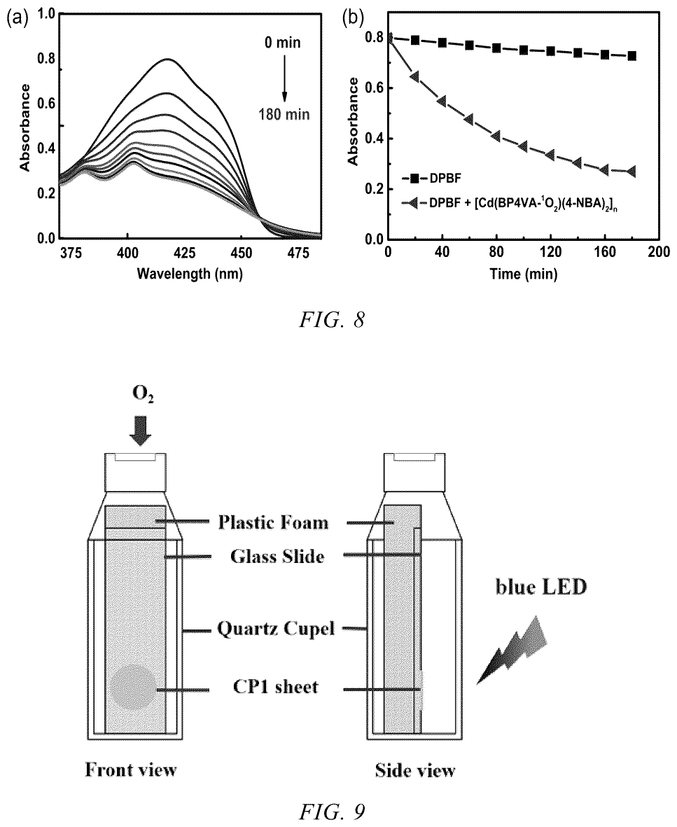

The reversible photo-oxidation process of [Cd(BP4VA-¹O₂)(4-NBA)₂]ₙ was studied. When heated with microwave, [Cd(BP4VA-¹O₂)(4-NBA)₂]ₙ could be completely converted into [Cd(BP4VA)(4-NBA)₂]ₙ in 9 min. 1,3-diphenyl isobenzofuran (DPBF) was used as a ¹O₂ capturing agent, to verify that ¹O₂ was released in the above process. A DPBF solution in dimethyl sulfoxide (DMSO) was added to [Cd(BP4VA-¹O₂)(4-NBA)₂]ₙ. By measuring the absorbance of DPBF at 416 nm, it was observed that DPBF decreased with the extension of heating time, until it was completely consumed, and an absorption peak of anthracene gradually appeared (FIG. 8). There is no obvious consumption of pure DPBF in the blank experiment (FIG. 8). This confirms that ¹O₂ is released during the heating of [Cd(BP4VA-¹O₂)(4-NBA)₂]ₙ. As shown in FIG. 8, the value of Ln(At/A0) has a good linear relationship with the heating time. This is a first-order kinetic process with a rate of 0.0065 min⁻¹. To quantify the ¹O₂ released by [Cd(BP4VA-¹O₂)(4-NBA)₂]ₙ, three capturing experiments were carried out with excessive DPBF. The average value shows that [Cd(BP4VA-¹O₂)(4-NBA)₂]ₙ releases about 57±3% ¹O₂.

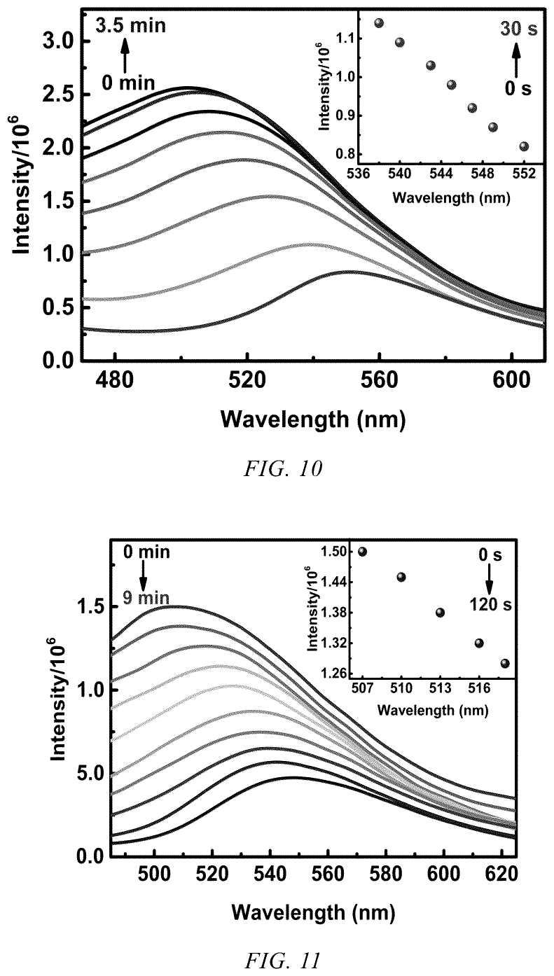

### Test Example 4. Measurement of Sensitivity in Fluorescence Response to O₂

[Cd(BP4VA)(4-NBA)₂]ₙ in Example 1 was used as a fluorescence sensing material to test its sensitivity in response to oxygen. The specific operation steps were shown in FIG. 9. The compound was made into a film and placed in a cuvette. Then, the gas introduced into the cuvette that is air or oxygen was adjusted by using a three-way valve, and the fluorescence intensity at 552 nm was tested. The results are shown in FIG. 10. The compound shows a rapid and sensitive response to oxygen, the response time is only within 5 s, and the recovery time is only about 30 s (FIG. 11). It can be seen that the compound shows good sensitivity in oxygen detection.

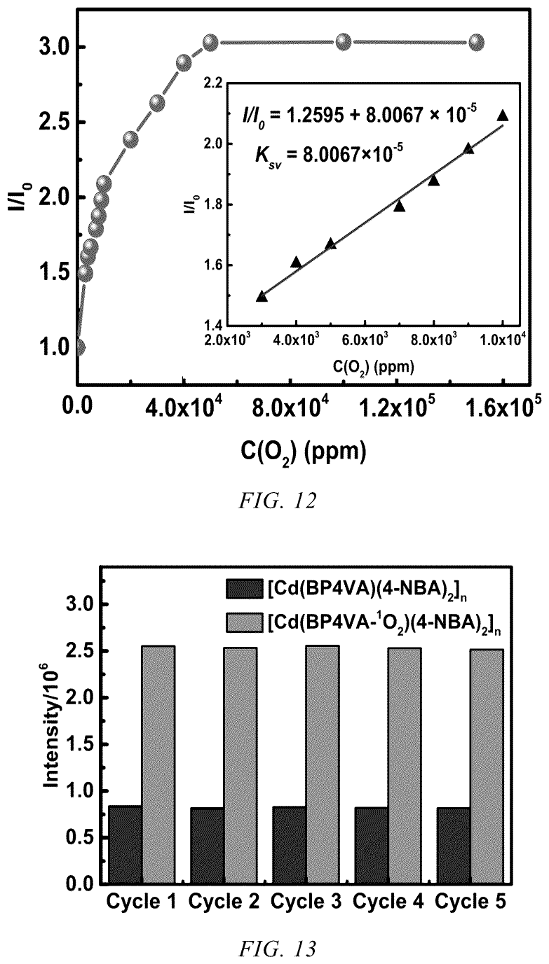

To determine the limit of detection of CP1 for oxygen, as shown in FIG. 12, when oxygen is at a low concentration (3×10³ to 1×10⁴ ppm), the luminous intensity-concentration relationship satisfies the linear equation I/I₀=1.25953+8.00674×10⁻⁵[M], where the Kₛᵥ constant is 8.00674×10⁻⁵. According to the definition 36/Kₛᵥ, the limit of detection (LOD) for oxygen is calculated to be 97.4 ppm, where δ is the standard deviation 0.0025990, which is calculated by measuring the average fluorescence intensity of the blank sample for 10 times.

### Test Example 5. Recyclability Test

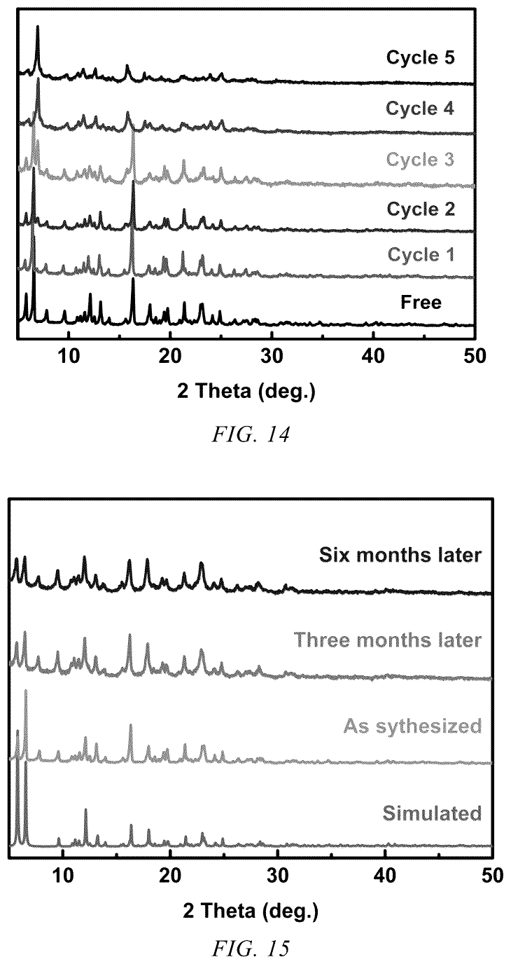

To test the recyclability of the compound in the fluorescence detection of oxygen, the instrument shown in FIG. 9 was used. The test results are shown in FIG. 13. The compound shows excellent recyclability in response to oxygen. After 5 cyclic tests, a high sensitivity is still maintained, and the fluorescence intensity before and after the response to oxygen has no obvious change. The results of PXRD analysis show that the compound remains in a good crystalline state after 5 reversible cycles. The reversible process of photo-oxidation can be repeated more than 5 times (FIG. 14).

### Test Example 6. Stability Test

After [Cd(BP4VA)(4-NBA)₂]ₙ was left in air for 6 months, the PXRD pattern (FIG. 15) shows that the structure remains stable, indicating the good stability of the compound. Apparently, the above-described embodiments are merely examples provided for clarity of description, and are not intended to limit the implementations of the present invention. Other variations or changes can be made by those skilled in the art based on the above description. The embodiments are not exhaustive herein. Obvious variations or changes derived therefrom also fall within the protection scope of the present invention.

What is claimed is:

1. A singlet oxygen capturing or releasing material, wherein the singlet oxygen capturing material has a chemical formula of [Cd(BP4VA)(4-NBA)₂]ₙ; and the singlet oxygen releasing material has a chemical formula of [Cd(BP4VA-¹O₂)(4-NBA)₂]ₙ, in which BP4VA is 9,10-bis[(cis)-2-(pyridin-4-yl)vinyl]anthracene, 4-NBA is a unit derived by removing a hydrogen ion from 4-nitrobenzoic acid after reaction, and n=3000-50000.

2. A method for preparing a singlet oxygen capturing or releasing material according to claim 1, comprising steps of:
S1: dissolving a soluble cadmium salt, 9,10-bis[(cis)-2-(pyridin-4-yl)vinyl]anthracene, and 4-nitrobenzoic acid in a solvent, reacting with heating, and filtering after the reaction, to obtain the singlet oxygen capturing material [Cd(BP4VA)(4-NBA)₂]ₙ; and
S2: subjecting the singlet oxygen capturing material obtained in S1 to a photo-oxidation reaction under an oxygen atmosphere, to obtain the singlet oxygen releasing material [Cd(BP4VA-¹O₂)(4-NBA)₂]ₙ;
in which BP4VA is 9,10-bis[(cis)-2-(pyridin-4-yl)vinyl]anthracene, 4-NBA is a unit derived by removing a hydrogen ion from 4-nitrobenzoic acid after reaction, and n=3000-50000.

3. The method for preparing a singlet oxygen capturing or releasing material according to claim 2, wherein in S1, the soluble cadmium salt is selected from the group consisting of cadmium nitrate, cadmium sulfate, cadmium perchlorate, cadmium carbonate and any combination thereof.

4. The method for preparing a singlet oxygen capturing or releasing material according to claim 2, wherein in S1, a molar ratio of the soluble cadmium salt, 9,10-bis[(cis)-2-(pyridin-4-yl)vinyl]anthracene and 4-nitrobenzoic acid is 1-1.5:0.75-1.5:1.5-2.5.

5. The method for preparing a singlet oxygen capturing or releasing material according to claim 2, wherein in S1, the reaction temperature is 120° C.-135° C., and the reaction time is 18-36 h.

6. The method for preparing a singlet oxygen capturing or releasing material according to claim 2, wherein in S1, the solvent comprises N,N-dimethyl acetamide, acetonitrile and water mixed in a volume ratio of 1.0-2.0:1.0-2.0:2.5-3.5.

7. The method for preparing a singlet oxygen capturing or releasing material according to claim 2, wherein in S2, a light source for the photo-oxidation reaction is visible light having a wavelength of 475 nm.

8. The method for preparing a singlet oxygen capturing or releasing material according to claim 2, wherein in S2, the photo-oxidation reaction time is 4-7 h.

9. A fluorescence sensor, comprising the singlet oxygen capturing material according to claim 1.

10. Use of the fluorescence sensor according to claim 9 in the detection of oxygen.
